SEQUENCE LISTING (8 sequences)

SEQ ID NO: 1 - DNA, 2169 bp, Homo sapiens
gccagagcggcgcggcctggaagaggccagggcccgggggaggcgacggcagcggcggcggctggggcagcccgggcagcccgagccccgcagcctgggcctgtgctcggcgccatgagcggcggcgggccttcgggaggcggccctgggggctcgggcagggcgcggactagctcgttcgcggagcccggcggcggaggcggaggaggcggcggcggccccggaggctcggcctccggcccaggcggcaccggcggcggaaaggcatctgtcggggccatgggtgggggcgtcggggcctcgagctccgggggtggacccggcggcagcggcggaggaggcagcggaggccccggcgcaggcactagcttcccgccgcccggggtgaagctgggccgtgacagcgggaaggtgaccacagtcgtagccactctaggccaaggcccagagcgctcccaagaagtggcttacacggacatcaaagtgattggcaatggctcatttggggtcgtgtaccaggcacggctggcagagaccagggaactagtcgccatcaagaaggttctccaggacaagaggttcaagaaccgagagctgcagatcatgcgtaagctggaccactgcaatattgtgaggctgagatactttttctactccagtggcgagaagaaagacgagctttacctaaatctggtgctggaatatgtgcccgagacagtgtaccgggtggcccgccacttcaccaaggccaagttgaccatccctatcctctatgtcaaggtgtacatgtaccagctcttccgcagcttggcctacatccactcccagggcgtgtgtcaccgcgacatcaagccccagaacctgctggtggaccctgacactgctgtcctcaagctctgcgattttggcagtgcaaagcagttggtccgaggggagcccaatgtctcctacatctgttctcgctactaccgggccccagagctcatctttggagccactgattacacctcatccatcgatgtttggtcagctggctgtgtactggcagagctcctcttgggccagcccatcttccctggggacagtggggtggaccagctggtggagatcatcaaggtgctgggaacaccaacccgggaacaaatccgagagatgaaccccaactacacggagttcaagttccctcagattaaagctcacccctggacaaaggtgttcaaatctcgaacgccgccagaggccatcgcgctctgctctagcctgctggagtacaccccatcctcaaggctctccccactagaggcctgtgcgcacagcttctttgatgaactgcgatgtctgggaacccagctgcctaacaaccgcccacttccccctctcttcaacttcagtgctggtgaactctccatccaaccgtctctcaacgccattctcatccctcctcacttgaggtcccccagcggcactaccaccctcaccccgtcctcacaagctttaactgagactccgaccagctcagactggcagtcgaccgatgccacacctaccctcactaactcctcctgagggccccaccaagcacccttccacttccatctgggagccccaagagggcgtgggaaggggggccatagcccatcaagctcctgccctggctgggcccctagactagagggcagaggtaaatgagtccctgtccccacctccagtccctccctcaccagcctcacccctgtggtgggctttttaagaggattttaactggttgtggggagggaagagaaggacagggtgttggggggatgaggacctcctacccccttggccccctcccctcccccagacctccacctcctccagaccccctcccctcctgtgtcccttgtaaatagaaccagcccagcccgtctcctcttcccttccctggcccccgggtgtaaatagattgttataatttttttcttaaagaaaacgtcgattcgcaccgtccaacctgccccgcccctcctacagctgtaactcccctcctgtcctctgcccccaaggtctactccctcctcaccccaccctggagggccaggggagtggagagagctcctgatgtcttagtttccacagtaaggtttgcctgtgtacagacctccgttcaataaattattggcatgaaaa

SEQ ID NO: 2 - protein, 483 aa, Homo sapiens
MSGGGPSGGGPGGSGRARTSSFAEPGGGGGGGGGGPGGSASGPGGTGGGKASVGAMGGGVGASSSGGGPGGSGGGGSGGPGAGTSFPPPGVKLGRDSGKVTTVVATLGQGPERSQEVAYTDIKVIGNGSFGVVYQARLAETRELVAIKKVLQDKRFKNRELQIMRKLDHCNIVRLRYFFYSSGEKKDELYLNLVLEYVPETVYRVARHFTKAKLTIPILYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFKSRTPPEAIALCSSLLEYTPSSRLSPLEACAHSFFDELRCLGTQLPNNRPLPPLFNFSAGELSIQPSLNAILIPPHLRSPSGTTTLTPSSQALTETPTSSDWQSTDATPTLTNSS

SEQ ID NO: 3 - DNA, 1389 bp, Homo sapiens
ggagaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgacgagccagctgcacaggaaaaaccaccagttacttgagtgtcactcagcaacactggtcacgtttggaaagaatatt

SEQ ID NO: 4 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKHPNGRDTPALFNFTTQELSSNPPLATILIPPHARIQAAASTPTNATAASDANTGDRGQTNNAASASASNST

SEQ ID NO: 5 - DNA, 1452 bp, Artificial Sequence
  Description of Artificial Sequence Coding nucleic acid sequence of the transgene used for the production of GSK-3 alpha transgenic non-human animals
atgagcggcggcgggccttcgggaggcggccctgggggctcgggcagggcgcggactagctcgttcgcggagcccggcggcggaggcggaggaggcggcggcggccccggaggctcggcctccggcccaggcggcaccggcggcggaaaggcatctgtcggggccatgggtgggggcgtcggggcctcgagctccgggggtggacccggcggcagcggcggaggaggcagcggaggccccggcgcaggcactagcttcccgccgcccggggtgaagctgggccgtgacagcgggaaggtgaccacagtcgtagccactctaggccaaggcccagagcgctcccaagaagtggcttacacggacatcaaagtgattggcaatggctcatttggggtcgtgtaccaggcacggctggcagagaccagggaactagtcgccatcaagaaggttctccaggacaagaggttcaagaaccgagagctgcagatcatgcgtaagctggaccactgcaatattgtgaggctgagatactttttctactccagtggcgagaagaaagacgagctttacctaaatctggtgctggaatatgtgcccgagacagtgtaccgggtggcccgccacttcaccaaggccaagttgaccatccctatcctctatgtcaaggtgtacatgtaccagctcttccgcagcttggcctacatccactcccagggcgtgtgtcaccgcgacatcaagccccagaacctgctggtggaccctgacactgctgtcctcaagctctgcgattttggcagtgcaaagcagttggtccgaggggagcccaatgtctcctacatctgttctcgctactaccgggccccagagctcatctttggagccactgattacacctcatccatcgatgtttggtcagctggctgtgtactggcagagctcctcttgggccagcccatcttccctggggacagtggggtggaccagctggtggagatcatcaaggtgctgggaacaccaacccgggaacaaatccgagagatgaaccccaactacacggagttcaagttccctcagattaaagctcacccctggacaaaggtgttcaaatctcgaacgccgccagaggccatcgcgctctgctctagcctgctggagtacaccccatcctcaaggctctccccactagaggcctgtgcgcacagcttctttgatgaactgcgatgtctgggaacccagctgcctaacaaccgcccacttccccctctcttcaacttcagtgctggtgaactctccatccaaccgtctctcaacgccattctcatccctcctcacttgaggtcccccagcggcactaccaccctcaccccgtcctcacaagctttaactgagactccgaccagctcagactggcagtcgaccgatgccacacctaccctcactaactcctcctga

SEQ ID NO: 6 - DNA, 1263 bp, Artificial Sequence
  Description of Artificial Sequence Coding nucleic acid sequence of the transgene used for the production of GSK-3 beta transgenic non-human animals
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 7 - DNA, 26 bp, Artificial Sequence
  Description of Artificial Sequence Primer
gctggaattaattcgctgtctgcgag

SEQ ID NO: 8 - DNA, 27 bp, Artificial Sequence
  Description of Artificial Sequence Primer
atgcatgctcgacctgcagttggaacc

(19) United States
(12) Patent Application Publication
(10) Pub. No.: US 2004/0261137 A1
(43) Pub. Date: Dec. 23, 2004

(54) Models for metabolic disorders

(76) Inventor: Julie Caroline Holder, Welwyn (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/482,524

(22) PCT Filed: Jun. 25, 2002
(86) PCT No.: PCT/GB02/02927

(30) Foreign Application Priority Data
Jun. 26, 2001 (GB) 0115570.4
Mar. 9, 2002 (GB) 0205604.2

(51) Int. Cl. A01K 67/027; C12N 5/06

(52) U.S. Cl. 800/14; 435/354

(57) ABSTRACT

A non-human animal model for screening potential agents for the treatment of diseases relating from changes in glucose homeostasis, such as diabetes and other metabolic diseases such as obesity is disclosed. The invention also relates to associated polynucleotide constructs, vectors, cells and methods for testing and screening such potential drugs.

### FIELD OF THE INVENTION

The present invention relates to a non-human animal model for screening potential agents for the treatment of diseases resulting from changes in glucose homeostasis, such as diabetes and other metabolic diseases such as obesity. The invention also relates to associated polynucleotide constructs, vectors, cells and methods for testing and screening such potential drugs.

### BACKGROUND OF THE INVENTION

Insulin stimulated glucose disposal is impaired in patients with type 2 diabetes. The primary site of glucose disposal is skeletal muscle, where glucose is either metabolised via glycolysis and the TCA cycle, or stored as glycogen. Since glycogen synthesis accounts for a large proportion of insulin stimulated glucose utilisation in skeletal muscle, the regulation of glycogen synthase (GS), the rate limiting enzyme in glycogen synthesis, has been the focus of much research. Several laboratories have demonstrated that the activity of GS and the rates of glycogen synthesis are reduced in skeletal muscles of type 2 diabetics (Shulman, N. Eng. J. Med., 322, 223, 1990, De Fronzo et al., Diabetes Care 15, 318, 1992, Thorburn et al., J. C. I. , 85, 522, 1990, Neilson et al., Diabetes Care 15, 418, 1992). Muscle glycogen synthesis thus provides an important metabolic route to store excess glucose and maintain blood glucose homeostasis.

The activity of GS is controled, at least in part, by phosphorylation of specific serine residues within the enzyme. The phosphorylation and consequent inactivation of GS is catalysed by the enzyme glycogen synthase kinase-3 (GSK-3), whereas dephosphorylation and consequent activation is catalysed by protein phosphatase-1 (PP1). Control of GS activity by PP-1 is not believed to be impaired in type 2 diabetes (Barriocanal et al., Diab. Med. 12, 1110, 1995). However, recent evidence indicates that the level of GSK-3 protein and its consequent total activity are elevated in skeletal muscle of type 2 diabetics (Nikoulina et al., Diabetes, 49, 263, 2000). Furthermore muscle GSK-3 levels were inversely correlated to both GS activity and insulin stimulated glucose disposal. Modulation of GSK-3 protein levels and/or activity may thus play an important role in the pathogenesis of diabetes.

GSK-3 is a serine/threonine protein kinase which phosphorylates specific serine residues in GS at serines with the consensus sequences SXXXS (Cohen, The Enzyme, XVIII, 461, 1986). There are 2 isoforms of the human enzyme currently known, α and β which appear to be ubiquitously expressed. It is possible that more isoforms exist that have not yet been identified. The alpha form (Mr 51 kDa) has an 85% homology with the smaller beta form (Mr 46 kDa) (Woodgett, EMBO Journal, 9, 2431, 1990). GSK-3 alpha and GSK-3 beta map to 19q 13.2 and 3q13.3 respectively (Shaw et al., Genome 41, 720, 1998. Their functional differences, if any, are unknown at this stage.

There is a need to characterise further the GSK-3 genes and the GSK-3 polypeptides expressed therefrom, to determine the function of these enzymes and to investigate the effect of increased or reduced expression and its relevance to disease. In addition the consequences of altered spatial or temporal expression of the GSK-3 enzymes need to be investigated as well as the effects of altered GSK-3 enzymes, where such alterations may have arisen through mutation for example. There is also a need to provide a means to identify and evaluate (with regard efficacy and safety) chemical compounds that modulate the activity of the GSK-3 genes or enzymes. Such modulation may, for example, be afforded by compounds which bind to and activate (agonist) or inhibit activation (antagonist) of the GSK-3 enzyme. Compounds identified thereby could be useful in therapy of diseases where inappropriate expression, or activation, of the GSK-3 enzyme or a mutated form of the GSK-3 enzyme is implicated or where GSK-3 enzyme modulates the activity of another molecule involved in the aetiology of the disease. There is a particular need to characterise further the human GSK-3 enzymes, because of their potential role in diabetes, obesity, neurological diseases, such as Alzheimers disease and stroke, and cardiovascular diseases (e.g. Coronary Heart Disease). One method for facilitating such studies is the generation and use of a transgenic rodent capable of expressing the human GSK-3 enzymes.

### SUMMARY OF THE INVENTION

The present inventors have generated a transgenic animal model of glucose intolerance. They have found that the use of a construct comprising a specific muscle promoter regulating the expression of the human GSK-3 enzyme results in a transgenic non-human animal which develops convincing glucose intolerance, and changes in glucose homeostasis. Such animals allow modulators of GSK-3 to be tested in vivo as possible treatments for diseases, primarily metabolic diseases, that are characterised by changes in glucose homeostasis. Accordingly, the present invention provides a transgenic non-human animal whose genome comprises a polynucleotide which encodes a human Glycogen Synthase Kinase-3 (GSK-3) polypeptide, said polynucleotide being under the control of a regulatory sequence which specifically directs expression of said GSK-3 polypeptide in muscle tissue.

### A BRIEF DESCRIPTION OF THE SEQUENCE LISTINGS AND FIGURES

SEQ ID NO:1 provides the nucleic acid sequence of the full GSK-3α gene including 5′ and 3′ untranslated regions.

SEQ ID NO:2 provides the amino acid sequence of the GSK-3α protein

SEQ ID NO:3 provides the nucleic acid sequence of the full GSK-3β gene including 5′ and 3′untranslated regions. SEQ ID NO:4 provides the amino acid sequence of the GSK-3β protein.

SEQ ID NO: 5 provides the coding nucleic acid sequence of the transgene used for the production GSK-3 α transgenic non-human animals.

SEQ ID NO: 6 provides the coding nucleic acid sequence of the transgene used for the production of GSK-3β transgenic non-human animals.

FIG. 1: This demonstrates the differences in body weights between GSK-3 transgenic mice and wild type control mice.

FIG. 2: This shows the results of a glucose tolerance test, highlighting the differences between the GSK-3β transgenic mice and the Wild type control mice.

FIG. 3: This shows the results of a glucose tolerance test, highlighting the differences between the GSK-3α transgenic mice and the Wild type control mice.

FIG. 4: This demonstrates phenotypic reversal by a GSK-3 inhibitor in GSK-3β transgenic mice.

FIG. 5: This demonstrates the expression of human GSK-3 β protein in the skeletal muscle of male (A) and female (B) transgenic mice.

FIG. 6: This demonstrates the Expression of human GSK-3α protein in the skeletal muscle of male (A) and female (B) transgenic mice

FIG. 7: This shows glycogen synthase activity in muscle and liver supernatants.

FIG. 8: This demonstrates the body composition of GSK-3, transgenic and control mice at 6 weeks of age (A) and 25 weeks of age (β)

FIG. 9: This demonstrates the tissue weights of GSK-3β transgenic and control mice aged 29 weeks.

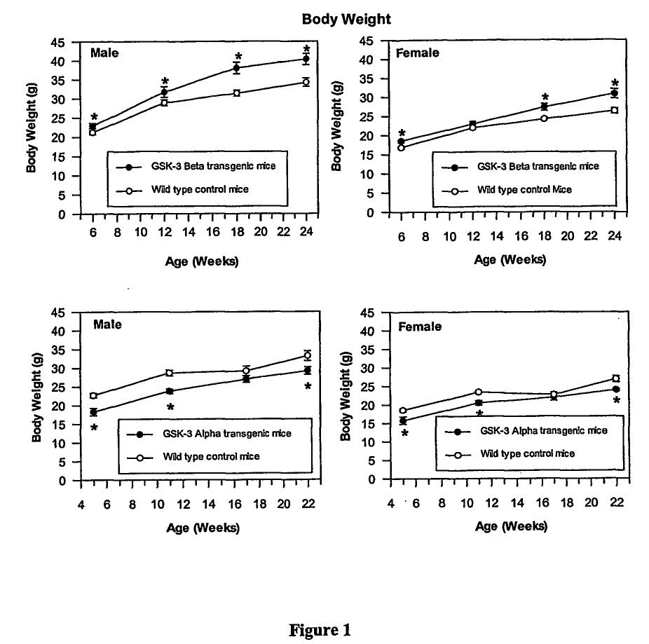

In FIGS. 1, 2, 3 and 4, the asterisks denotes a statistically significant difference between the transgenic animal and the wild type controls.

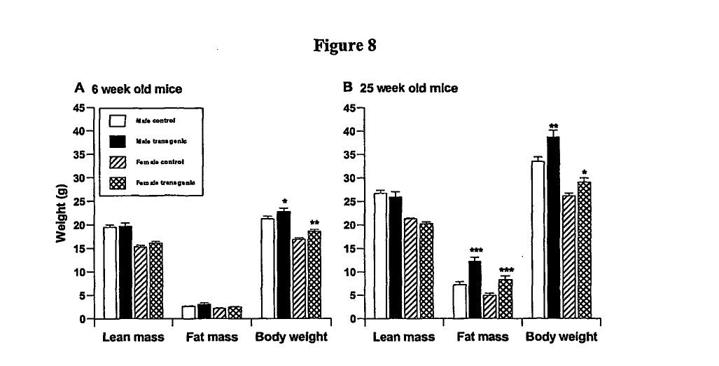

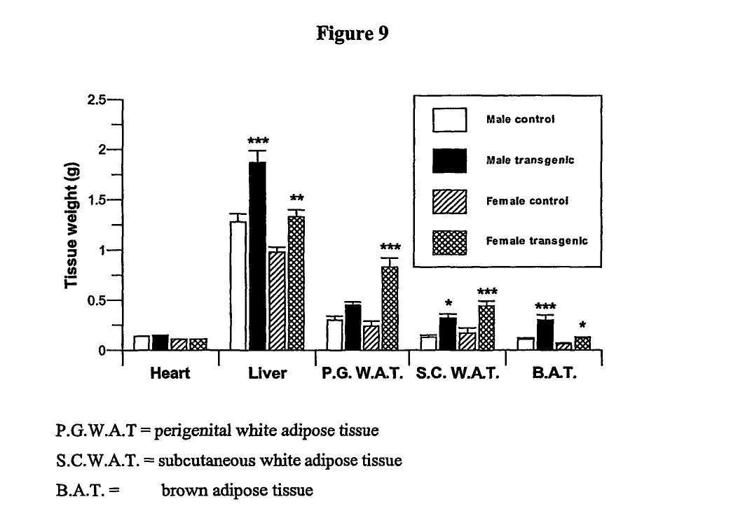

In FIGS. 8 and 9, * indicates P<0.05, ** indicates P<0.01 and *** indicates P<0.0001.

### DETAILED DESCRIPTION OF THE INVENTION

The present invention provides a transgenic non-human animal whose genome comprises a polynucleotide encoding one or more human GSK-3 polypeptides under the control of a regulatory sequence which specifically directs expression of said polypeptides in muscle. By the term “GSK-3 polypeptide” is meant a polypeptide that has the basic biological functionality of the human GSK-3 enzyme. Such basic biological functionality would be appreciated by a person skilled in the art to mean the ability to phosphorylate substrates of the GSK-3 enzyme such as the Glycogen Synthase protein/enzyme. The term GSK-3 polypeptide includes the wild type form of the GSK-3 enzyme, as well as modified forms of the GSK-3 enzyme, for example by the addition, substitution or deletion of one or more amino acids, or indeed fragments of the GSK-3 enzyme, provided that such modified forms or fragments have the basic biological functionality of the wild type GSK-3 enzyme.

The GSK-3 enzyme may be found in different isoforms, and the term GSK-3 polypeptide includes all isoforms of the GSK-3 enzyme. In one aspect, the GSK-3 polypeptide is the GSK-3α isoform. In another aspect, the GSK-3 polypeptide is the GSK-3β isoform.

In a preferred aspect the transgenic non-human animal is a mammal, preferably a rodent. Preferably, it is a mouse or a rat, and particularly preferably it is a mouse. The polynucleotide encoding one or more GSK-3 polypeptides comprised in the genome of a transgenic non-human animal of the invention is selected from the following group when one or more of said polypeptides are the GSK-3α isoform:

(a) a polynucleotide comprising a polynucleotide sequence having at least 95%, 96%, 97%, 98%, or 99% identity to the polynucleotide sequence of SEQ ID NO:1 and/or SEQ ID NO:5;

(b) a polynucleotide comprising the polynucleotide sequence of SEQ ID NO:1 or SEQ ID NO:5;

(c) a polynucleotide having at least 95%, 96%, 97%, 98%, or 99% identity to the polynucleotide of SEQ ID NO:1 and/or SEQ ID NO:5;

(d) the polynucleotide of SEQ ID NO:1 or SEQ ID NO:5;

(e) a polynucleotide comprising a polynucleotide sequence encoding a polypeptide sequence having at least 95%, 96%, 97%, 98%, or 99% identity to the polypeptide sequence of SEQ ID NO:2;

(f) a polynucleotide comprising a polynucleotide sequence which encodes the polypeptide of SEQ ID NO:2;

(g) a polynucleotide having a polynucleotide sequence which encodes a polypeptide sequence having at least 95%, 96%, 97%, 98%, or 99% identity to the polypeptide sequence of SEQ ID NO:2;

(h) a polynucleotide which encodes the polypeptide of SEQ ID NO:2;

(i) a polynucleotide having or comprising a polynucleotide sequence that has an Identity Index of 0.95, 0.96, 0.97, 0.98, or 0.99 compared to the polynucleotide sequence of SEQ ID NO:1 and/or SEQ ID NO:5;

(j) a polynucleotide having or comprising a polynucleotide sequence encoding a polypeptide sequence that has an Identity Index of 0.95, 0.96, 0.97, 0.98, or 0.99 compared to the polypeptide sequence of SEQ ID NO:2;

(k) a polynucleotide fragment of SEQ ID NO:1 or SEQ ID NO:2 that encodes a functional GSK-3 polypeptide;

(l) a polynucleotide that will hybridise under stringent conditions to a polynucleotide of SEQ ID NO:1 and/or SEQ ID NO:5 and that encodes a functional GSK-3 polypeptide.

The polynucleotide encoding one or more GSK-3 polypeptides comprised in the genome of a non-human animal of the invention is selected from the following group when one or more of said polypeptides is the GSK-3β isoform:

(a) a polynucleotide comprising a polynucleotide sequence having at least 95%, 96%, 97%, 98%, or 99% identity to the polynucleotide sequence of SEQ ID NO:3 and/or SEQ ID NO:6;

(b) a polynucleotide comprising the polynucleotide of SEQ ID NO:3 or SEQ ID NO:6;

(c) a polynucleotide having at least 95%, 96%, 97%, 98%, or 99% identity to the polynucleotide of SEQ ID NO:3 and/or SEQ ID NO:6;

(d) the polynucleotide of SEQ ID NO:3 or SEQ ID NO:6;

(e) a polynucleotide comprising a polynucleotide sequence encoding a polypeptide sequence having at least 95%, 96%, 97%, 98%, or 99% identity to the polypeptide sequence of SEQ ID NO:4;

(f) a polynucleotide comprising a polynucleotide sequence encoding the polypeptide of SEQ ID NO:4;

(g) a polynucleotide having a polynucleotide sequence encoding a polypeptide sequence having at least 95%, 96%, 97%, 98%, or 99% identity to the polypeptide sequence of SEQ ID NO:4;

(h) a polynucleotide encoding the polypeptide of SEQ ID NO:4;

(i) a polynucleotide having or comprising a polynucleotide sequence that has an Identity Index of 0.95, 0.96, 0.97, 0.98, or 0.99 compared to the polynucleotide sequence of SEQ ID NO:3 and/or SEQ ID NO:6;

(j) a polynucleotide having or comprising a polynucleotide sequence encoding a polypeptide sequence that has an Identity Index of 0.95, 0.96, 0.97, 0.98, or 0.99 compared to the polypeptide sequence of SEQ ID NO:3 and/or SEQ ID NO:6;

(k) a polynucleotide fragment of SEQ ID NO:3 or SEQ ID NO: 6 which encodes a functional GSK-3 polypeptide

(l) a polynucleotide that will hybridise under stringent conditions to a polynucleotide of SEQ ID NO:3 and/or SEQ ID NO: 6 and which encodes a functional GSK-3 polypeptide.

The polynucleotides included in the groups above are herein termed GSK-3 encoding polynucleotides.

The polynucleotide comprised in the genome of the transgenic non-human animal may comprise any number of nucleic acid sequences encoding any type of isoform. For example, the polynucleotide may comprise one or more nucleic acid sequences encoding the GSK-3α isoform. Alternatively, the polynucleotide may comprise one or more nucleic acid sequences encoding the GSK-3β isoform. In a further aspect, the polynucleotide may comprise one or more nucleic acid sequences encoding the GSK-3α isoform, and additionally one or more nucleic acid sequences encoding the GSK-3βisoform. In all aspects, the polynucleotide is under the control of a regulatory sequence that directs expression of the GSK-3 polypeptides in muscle. Where the polynucleotide comprises multiple nucleic acid sequences encoding GSK-3 polypeptides, one or more of said nucleic acid sequences may be under the control of a single regulatory sequence, or each nucleic acid sequence may be under the control of a separate regulatory sequence, which regulatory sequences may be identical or different.

The present invention relies on the use of a nucleic acid construct to generate a transgenic animal model for screening agents for potential use in the treatment of diseases related to changes in glucose homeostasis, such as diabetes, obesity and atherosclerosis. The construct comprises a GSK-3 encoding polynucleotide as defined herein and a muscle specific regulatory sequence such as, for example an α-skeletal actin promoter, a gamma-sarcoglycan promoter, a rat phosphoglycerate mutase m gene promoter, a beta-myosin heavy chain promoter, a Muscle creatine kinase promoter or a myogenin promoter. Preferably, the regulatory sequence is a skeletal muscle specific regulatory sequence. Particularly preferred is when the regulatory sequence comprises an α-skeletal actin promoter. By muscle specific is meant that the regulatory sequence will direct expression of the GSK-3 encoding polynucleotide primarily in muscle cells only. A person skilled in the art will appreciate that there may be some expression in other cells due to a small amount of non-specific expression. Preferably, at least 80%, 85% or 90% of the total expression of the transgene is within muscle cells. Further preferred is when at least 90%, for example 95%, 96%, 97% 98% or 99% of the total expression of the transgene is within muscle cells. Particularly preferred is when 100% of the expression of the transgene is within muscle cells.

The regulatory sequence may be from any organism, but preferably it is mammalian, and particularly preferably it is from a rodent such as a rat or a mouse. The assembly of the transgenic construct follows standard cloning techniques, that are well known in the art (for example see Sambrook et al., Molecular Cloning: A Laboratory Manual, 2nd Ed., Cold Spring Harbor Laboratory Press, Cold Spring Harbor, N.Y. (1989)). The GSK-3 encoding polynucleotide may consist of cDNA or genomic DNA. If it is cDNA, the cDNA to be overexpressed can be prepared from a mRNA extracted from a relevant tissue, preferably a tissue in which the GSK-3 polypeptide is known to be expressed. Should the GSK-3 encoding polynucleotide encode a specific isoform, then the cDNA may be prepared from mRNA extracted from a tissue in which that isoform is known to be expressed. The cDNA, along with the muscle specific regulatory sequence of choice and any other desired components such as artificial introns and reporter genes, can then be inserted into a cloning vector by restriction digest and ligation. Suitable cloning vectors for the assembly of transgenes are those which provide for acceptable yields of DNA. Vectors such as pBluescript are particularly preferred as in addition to good yield, they provide desirable unique restriction sites flanking the transgene (for example BssHII in pBluescript) for convenient removal of the vector portion of the construct prior to pronucleus injection. In this instance, the desired regulatory sequences will need to be engineered into the vector. Alternatively, another preferred vector is the pACT-IVSpolyA vector, as this already contains within it human α-actin promoter sequences that can be used to drive skeletal muscle specific expression.

The regulatory sequences, such as promoters, are operably linked to the coding sequence of the GSK-3 encoding polynucleotide or polynucleotides in a manner that will permit the required temporal and spatial expression of the polynucleotide. There may or may not be intervening sequences between the linked polynucleotide or polynucleotides and the promoter, provided that the promoter directs expression of the polynucleotide or polynucleotides so linked to it Methods of so linking regulatory sequences to cDNAs to facilitate their expression are widely known in the art. Such methods include, for example, directly ligating a nucleic acid sequence comprising a regulatory sequence to the coding region of the GSK-3 encoding polynucleotide. Additional nucleic acid sequences may be included that modulate expression in the required manner. Examples of additional sequences include enhancer elements, artificial introns and others.

In addition the nucleotide sequence of a known promoter, or other regulatory sequence, may be modified to increase levels of expression. Such modifications can be achieved using, for example, site-directed mutagenesis methods well known in the art (see Sambrook et al, supra).

In addition to modifying the sequence of regulatory elements to enhance, or otherwise change, expression levels, the coding sequence of the GSK-3 encoding polynucleotide may be modified to enhance or otherwise affect expression levels. For example if the transgene is from a different species than the host, the codon usage of the transgene can be altered to match more closely that of the host. It is well known in the art that different organisms use the 64 coding and stop codons at different frequencies. Codons that are infrequently used in an organism are termed “rare codons”. If a transgene includes a codon that is a rare codon in the host, expression levels may be severely reduced. One solution is to replace one or more rare codons in the transgene with codons that are frequently used in the host. Other modifications to the transgene sequence include modifying the polynucleotide sequence surrounding the start codon (the initiator methionine encoding codon) to make this more closely match the consensus “Kozak” sequence (A/G CCATGG, where the ATG in bold is the start codon; see for example Kozak, M., Nucleic Acids Res (1984) May 11;12(9):3873-3893)). In the transcribed mRNA molecule the Kozak sequence is believed to provide the optimal environment for initiation of translation of the polypeptide.

Preferably, prior to the introduction of the transgene into the host cell, the vector portions are removed by restriction enzyme digestion, for example by using restriction sites in the vector that flank the transgene. Thus the genetic material that is actually introduced into the host cell will preferably comprise the coding sequence of the GSK-3 encoding polynucleotide and the regulatory sequences to which it has been operably linked together with other potential components of the transgene, for example a reporter gene. More preferably the genetic material will have only the transgene and the regulatory sequences to which it has been operably linked.

This invention also includes any cells cultured from the transgenic non-human animal. The cells are cultured in-vitro. The genome of the cells can thus comprise the construct of the invention. The GSK-3 polypeptide is typically expressed in such cells in an amount at least 2, preferably at least 5, for example 6, 7, 8, 9, and particularly preferably at least 10 times greater than the amount of endogenous GSK-3 polypeptide that is expressed in the cells.

Cells cultured in-vitro from a transgenic animal may be prepared by any suitable method. The cells are typically rodent and preferably mouse cells. Cultures of muscle cells can therefore be provided, for example cultures of skeletal or smooth muscle cells. The cells may be used to introduce other genes of interest by any known method (including viral delivery).

The invention also includes any cell transformed or transfected with a GSK-3 encoding polynucleotide of the invention. Such cells include bacterial and yeast cells used to replicate a vector comprising the GSK-3 encoding polynucleotide and stem cells or oocytes that are used to generate the non-human transgenic animals of the invention.

The non-human transgenic animals of the invention may be generated by the use of any appropriate protocol. A suitable method comprises:

Making a suitable cell of the invention;

allowing the cell to develop into an animal of the invention; and

optionally, breeding the animal true.

There are a number of techniques that permit the introduction of genetic material, such as a transgene, into the germline. The most commonly used, and preferred protocol comprises direct injection of the transgene into the male pronucleus of the fertilised egg (Hogan et al., Manipulating the mouse embryo (A laboratory manual) Second edition, CSHL Press 1994), resulting in the random integration into one locus of a varying number of copies, usually in a head to tail array (Costantini and Lacy, Nature 294, 92, 1981). The injected eggs are then re-transferred into the uteri of pseudo-pregnant recipient mothers. Some of the resulting offspring may have one or several copies of the transgene integrated into their genomes, usually in one integration site. These “founder” animals are then bred to establish transgenic lines and to back-cross into the genetic background of choice. It is convenient to have the transgene insertion on both chromosomes (homozygosity) as this obviates the need for repeated genotyping in the course of routine mouse husbandry.

Alternatively, for the production of transgenic mice, transgenes can be introduced via embryonic stem (ES) cells, using electroporation, retroviral vectors or lipofection for gene transfer. This is followed by the random insertion into the genome of the pluripotent embryonic stem (ES) cells, followed by the production of chimeric mice and subsequent germline transmission. Transgenes of up to several hundred kilobases of rodentian DNA have been used to produce transgenic mice in this manner (for example Choi et al., Nature Genet. 4, 117-123 (1993); Strauss et al., Science 259, 1904-07 (1993)). The latter approach can be tailored such that the transgene is inserted into a pre-determined locus (non-randomly, for example ROSA26 or HPRT) that supports ubiquitous as well as tissue specific expression of the transgene (Vivian et al., BioTechniques 27, 154-162 (1999)). The transgenic animals can be subsequently tested to ensure the required genotypic change has been effected, in any suitable fashion. This can be done by, for example, detecting the presence of the transgene by PCR with specific primers, or by Southern blotting of tail DNA with a specific probe. Testing for homozygosity of the transgene insertion may be carried out using quantitative Southern blotting to detect a twofold difference in signal strength between hetero- and homozygous transgenic animals. Once the desired genotype has been confirmed the transgenic animal line can be subjected to various tests to determine the phenotype. The tests involved in this phenotypic characterisation depend on what genotypic change has been effected, and may include, for example, morphological, biochemical and behavioural studies. The transgenic animals of the present invention demonstrate changes in glucose homeostasis and body weight gain, in particular glucose intolerance, and the effect of the overexpression of the human GSK-3 polypeptide may be demonstrated by any suitable test for this phenotype. Suitable tests are based on the hypothesis that GSK-3 enzyme controls the rate of muscle glycogen synthesis via phosphorylation and inactivation of glycogen synthase enzyme. Thus, simple measurements like weight gain, food intake, water intake, oral glucose tolerance tests and measurement of body composition are preferred phenotypic tests for the initial analysis of the GSK-3 transgenic non-human animals. Subsequently, other parameters of glucose homeostasis and body composition can be investigated. Based on the results of these studies, further, more specific tests can be devised to give a more detailed analysis of the consequences of GSK-3 expression.

GSK-3 also has other functions within cells , such as regulation of Tau (Sperber et al., Neurosci. Lett., 197, 149-153, (1995)), IRS-1 (Eldar-Finkelman and Krebs, Proc. Natl. Acad. Sci. 94, 9660-9664 (1997)), the translation initiation factor eIF2B (Welch et al., FEBS Lett., 421, 125-130, (1998)), transcription factors c-jun (Nikoulakaki et al., Oncogene, 8, 833-840, (1993)), CREB (Fiol et al., J. Biol. Chem. 269, 32187-32193, (1994)), NFAT (Beals et al., Science 275, 1930-1934, (1997)), β-catenin (Hart et al., Curr. Biol. 8, 573-581, (1998)), and C/EBPα Ross et al., Mol. Cell. Biol. 19, 8433-8441 (1999)). It is possible that the overexpression of GSK-3 polypeptide in muscle in the animals of the present invention may also lead to the development of other phenotypes related to the activation or inhibition of these regulatory proteins by GSK-3. Animals that develop such phenotypes could be useful in the screening methods described herein to identify modulators that reverse or enhance said phenotype.

Comparison of the phenotypes of a GSK-3 alpha non-human transgenic animal and a GSK-3 beta non-human transgenic animal could be used to elucidate and define any functional differences between these two isoforms.

The use of non-human animal models which overexpress or lack more than one gene can provide important insights into the interaction of different genetic loci in particular diseases, for example diabetes (Mauvais-Jarvis and Kahn, Diabetes and Metabolism, 26, 433-448, 2000). Consequently the interbreeding of the GSK-3 transgenic non-human animals of the present invention with non-human animal models which overexpress or underexpress a different gene may produce alternative and potentially superior animal models of diseases such as diabetes. For example the cross-breeding of GSK-3 transgenic mice of the invention with GLUT4 knockout mice (which are insulin resistant and for which the GLUT4 heterozygotes go on to develop diabetes (Shepherd and Kahn N. Eng. J. Med. 341, 248-257, 1999, Stenbit et al, Nat. Med., 3, 1096-1101, 1997)), may produce an alternative and potentially superior animal model of diabetes. Cross-breeding of the non-human GSK-3 transgenic animals of the invention with other transgenics or knockouts could also enable the identification of genes which pre-dispose for or protect against diseases such as diabetes.

Cells and animals of this invention may also be used to identify and test the efficacy of potential therapeutic agents for the treatment of conditions which may be caused by overexpression of GSK-3 polypeptides in muscle, for example diseases characterised by changes in glucose homeostasis such as metabolic diseases including diabetes, obesity and cahexia. Potential therapeutic agents may inhibit (antagonise) or promote (agonise) the activity of the GSK-3 polypeptide, and may exert effects at only one isoform, or may agonise or antagonise all forms of the enzyme. Preferably, such therapeutic agents will antagonise the GSK-3 alpha and/or the GSK-3 beta polypeptides.

A method of identifying a therapeutic agent for the treatment of a condition characterised by a change in glucose homeostasis can therefore be provided, comprising:

administering to an animal of the invention a candidate test substance and

determining whether the candidate substance (i) prevents or delays the onset of the condition or (ii) treats the condition.

Option (ii) may be tested by determining whether the candidate substance causes a decrease in any of the cellular or physiological changes caused by the condition. Such cellular or physiological changes may include changes in muscle glycogen synthase activity or glycogen or fat storage or changes in glucose tolerance.

A method of identifying a therapeutic agent for the treatment of a condition characterised by a change in glucose homeostasis can also be provided, comprising:

contacting a candidate substance with a cell of the invention, and

determining whether the candidate substance (i) prevents or delays the onset of cellular changes associated with the condition, or (ii) causes a decrease in any of the cellular changes caused by the condition (such as any of the cellular changes mentioned herein).

Suitable candidate substances which may be tested in the above methods include antibody products (for example, monoclonal and polyclonal antibodies, single chain antibodies, chimeric antibodies and CDR grafted antibodies). Furthermore, combinatorial libraries, defined chemical identities, small molecules, peptide and peptide mimetics, oligonucleotides and natural product libraries, such as display libraries (e.g. phage display libraries) may also be tested. The candidate substances may be chemical compounds. Batches of the candidate substances may be used in an initial screen of, for example, ten substances per reaction, and the substances of batches which show inhibition tested individually.

The transgenic non-human animal models of the invention may also be used to test the efficacy of potential therapeutic agents aimed at inhibiting downstream events of changes in glucose homeostasis, for example effects in the Central Nervous System, or hyperlipidaemia leading, for example, to atherosclerosis.

Agents which may reverse the changes in glucose homeostasis seen in the transgenic animals of the invention may be tested in-vivo or in cells or tissue preparations in-vitro. Compounds can be tested using the assays and tests used to characterise the invention. For example, after administration of any potential therapeutic agent, the response of the transgenic animal may be assessed by, for example, looking for an improvement in glucose tolerance or body weight as described in the Examples. Methods for screening potential therapeutic agents using cell lines or animals are established. ELISA or Homogenous Time Resolved Fuorescence (HTRF) methodologies can be used.

Agents identified in the screening methods of the invention may be used to prevent or treat the diseases discussed above. The condition of a patient suffering from such a disease can therefore be improved by administration of such a product. As discussed further in example (4), the present inventors have shown that the administration to a non-human transgenic animal of the invention of an inhibitor of GSK-3 reverses the disease phenotype of glucose intolerance. This demonstrates that modulators of the GSK-3 enzyme can be used to prevent or treat the diseases discussed above, and the invention accordingly includes such modulators. A therapeutically effective non-toxic amount of such an agent may be given to a human patient in need thereof.

The formulation of the product for use in preventing or treating the disease will depend upon factors such as the nature of the agent identified and the disease to be prevented or treated. Typically the agent is formulated for use with a pharmaceutically acceptable carrier or diluent. For example it may be formulated for intracranial, parenteral, intravenous, intramuscular, subcutaneous, transdermal or oral administration. A physician will be able to determine the required route of administration for each particular patient. The pharmaceutical carrier or diluent may be, for example, an isotonic solution.

The dose of product may be determined according to various parameters, especially according to the substance used; the age, weight and condition of the patient to be treated; the route of administration; and the required regimen. A suitable dose may however be from 0.1 to 100 mg/kg body weight such as 1 to 40 mg/kg body weight. Again, a physician will be able to determine the required route of administration and dosage for any particular patient.

The construct, cells or therapeutic agents of the invention may be present in a substantially isolated form. It will be understood that they may be mixed with carriers or diluents which will not interfere with their intended purpose and still be regarded as substantially isolated. Thus the construct, cell or therapeutic agent of the invention may also be in a substantially purified form, in which case it will generally comprise more than 90%, e.g. more 95%, 98% or 99%, by weight of the relevant preparation.

Transgenic non-human animals which overexpress GSK-3α can be crossed with transgenic non-human animals which overexpress GSK-3β to produce homozygotes expressing both human GSK-3 α and GSK-3 β. These animals could also be used as described above.

The following definitions are provided to facilitate understanding of certain terms used frequently herein before.

A “Transgene” comprises a polynucleotide, isolated from nature, which has been manipulated in-vitro and subsequently introduced into the genome of the same or a different species in either the native or modified forms, such that it is stably and heritably maintained in that genome. Native forms include unmodified polynucleotides isolated from. a source different to that into which it is subsequently introduced, for example a human polynucleotide sequence introduced into a mouse genome. Modified polynucleotides include those which have one or more nucleotide substitutions, deletions, insertions or inversions. Native or modified polynucleotides may be operably linked to a heterologous promoter, or other regulatory sequence, from a different gene within the same species or from a gene in a different species. A polynucleotide is operably linked to a regulatory sequence when, for example, it is placed under the transcriptional control of said regulatory sequence. The polynucleotide may or may not encode a polypeptide, and if a polypeptide is expressed from the polynucleotide, said polypeptide may or may not be full-length relative to that encoded by the original polynucleotide isolated. The term transgene is generally used to refer to the polynucleotide and the regulatory sequences to which it is operably linked.

An organism into which a transgene has been introduced is termed a “transgenic” organism.

“Regulatory sequences” refer to DNA or RNA polynucleotide sequences, which are usually non-coding, that are involved in the regulation of transcriptional activity or tissue-specific enhancement or silencing of gene transcription. Such regulatory sequences include promoters and enhancers.

“Identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al, J. Mol. Biol. 215: 403-410 (1990). The well known Smith Waterman algorithm may also be used to determine identity.

Preferred parameters for polypeptide sequence comparison include the following:

1) Algorithm: Needleman and Wunsch, J. Mol. Biol. 48: 443453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parametersare the default parameters for peptide comparisons (along with no penalty for end gaps).

Preferred parameters for polynucleotide comparison include the following:

1) Algorithm: Needleman and Wunsch, J. Mol. Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity(divided by 100) and subtracting that product from said total number of nucleotides in SEQ ID NO:1, or:

nₙ≦xₙ−(xₙ•y),

wherein nₙ is the number of nucleotide alterations, xₙ is the total number of nucleotides in SEQ ID NO:1, and y is, for instance, 0.70 for 70%, 0.80 for 80%, 0.85 for 85%, 0.90 for 90%, 0.95 for 95%,etc., and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity(divided by 100) and then subtracting that product from said total number of amino acids in SEQ ID NO:2, or:

nₐ≦xₐ−(xₐ•y),

wherein nₐ is the number of amino acid alterations, xₐ is the total number of amino acids in SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ.

### EXAMPLES

### Example 1

### Preparation of GSK3 Alpha and GSK3 Beta Expression Vectors

1.1.

Human GSK-3 alpha cDNA was amplified by PCR using Pfu polymerase (Stratagene), from a cDNA comprising the polynucleotide sequence of SEQUENCE NO:1 as template. The PCR product was sequenced and cloned into pCR2.1(Strategene) by a blunt end ligation.

1.2.

Human GSK-3 beta cDNA was amplified by PCR using Pfu polymerase (Stratagene), from a cDNA comprising the polynucleotide sequence of SEQUENCE NO:3 as template. The PCR product was sequenced and cloned into pCR2.1 (Strategene) by a blunt end ligation.

1.3.

The bacterial betagalactosidase (LacZ) cDNA was isolated from the plasmid pIND/lacZ as an NheI-EcoR1 restriction fragment, and blunt end cloned into pBluescript SK(+) (Stategene) that had been digested with SpeI and EcoR1 and then blunt end filled.

1.4.

An artificial intron sequence was amplified by PCR using Pfu polymerase, with pRES1neo (Clontech) as template and the following primer pair: 5′ GCTGGAATTAATTCGCTGTCTGCGAG 3′ and 5′ ATGCATGCTCGACCTGCAGTTGGAACC 3′. The PCR fragment was then cloned into the XhoI and SfiI sites of pCEP4 (Invitrogen), via blunt end ligation.

1.5.

The artificial intron-SV40 polyA cassette was excised from the vector of 1.3 as a SalI-XhoI fragment and cloned into the XhoI site of the vector of 1.2 downstream of the LacZ cDNA.

1.6.

The human skeletal muscle specific alpha-actin promoter was excised from vector pACTSV40 (Fazeli et al (1996) J Cell Biol, 135 p241-251) as a 2.2 kb HindIII fragment, and cloned into the unique SmaI site of the vector of 1.4 via blunt end ligation to generate pACTlacZIVSpolyA.

1.7.

The vector of 1.6 was digested with EcoR1 and Cla1 to remove the LacZ cDNA sequences and the molecule was then end filled. Into this molecule was blunt end cloned the GSK-3 alpha cDNA sequence that was removed from the plasmid of 1.1 by digestion with EcoR1 and blunt end filled prior to cloning.

1.8.

The vector of 1.6 was digested with EcoR1 and Cla1 to remove the LacZ cDNA sequences and the molecule was then end filled. Into this molecule was blunt end cloned the GSK-3-beta cDNA sequence that was removed from the plasmid of 1.2 by digestion with EcoR1 and blunt end filled prior to cloning.

### Example 2

### Production of GSK-3α and GSK-3β Overexpressing Mice

The GSK-3 alpha expressing transgene DNA was excised from the vector of 1.7 (in example 1, above) with NotI and a partial Kpn1 restriction enzyme digests, gel-purified as a 4.4 kb kb DNA fragment, and injected into male pronuclei of fertilised eggs The GSK-3 beta expressing transgene DNA was excised from the vector of 1.8 (in example 1, above) with NotI and Kpn1 restriction enzyme digests, gel-purified as a 4.4 kb kb DNA fragment, and injected into male pronuclei of fertilised eggs.

To confirm that animals were overexpressing GSK-3 polypeptide, skeletal muscle samples (mixed fiber gastroc/plantaris) were removed, freeze-clamped in liquid nitrogen and stored at −80° C. Frozen muscle samples were ground to a fine powder under liquid nitrogen prior to homogenisation in buffer (50 mM Tris-HCl, pH7.5, 10 mM NaF, 5 mM Na₄P₂O₇, 1 mM Na₃VO₄, 10 mM β-glycerophosphate, 1 mM EDTA, 1 mM EGTA, 0.1% β-mercaptoethanol, 0.27M sucrose and protease inhibitor cocktail (‘Complete’, Boehringer Mannheim)). Triton-X-100 was added to 0.5% (v/v) and the homogenates were centrifuged at 10,000×g for 20 minutes at 4° C. Supernatants were removed and assayed for protein content prior to being stored at −80° C.

Protein samples were resolved by SDS-PAGE (30 ug of total protein per lane, 4-12% gradient gel) and transferred to PVDF membrane. Non-specific binding sites were blocked by incubation in TBS-tween containing 5% non-fat dry milk. GSK-3β was detected by incubation with a monoclonal anti-GSK-3β antibody (Transduction Laboratories, Cat. No.G22320) followed by an anti-mouse HRP conjugated secondary antibody (Amersham) and visualised using Supersignal West Dura (Pierce) chemiluminescent substrate. GSK-3α was detected using a rabbit polyclonal anti-GSK-3α antibody (Santa Cruz, Cat. No. sc-7879).

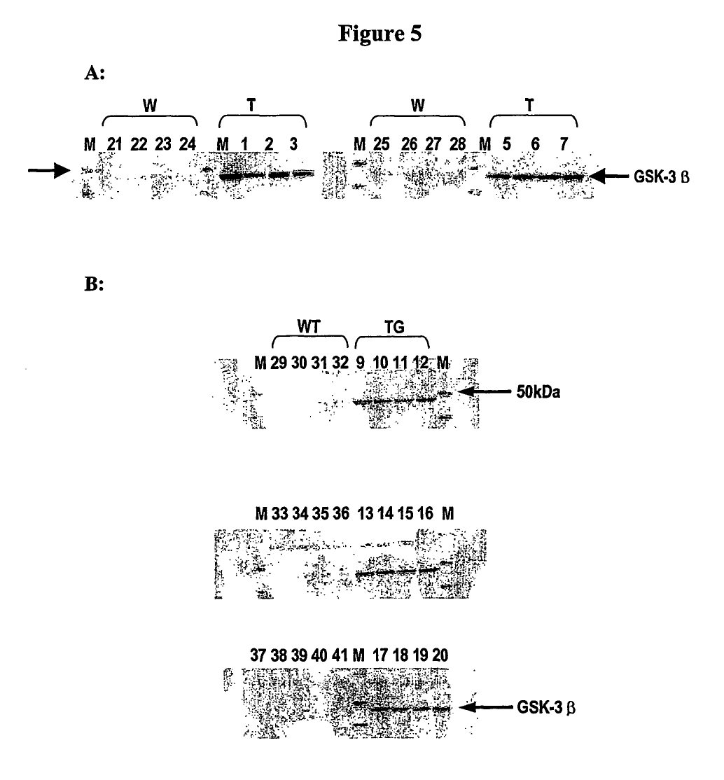

FIG. 5 shows immunoblots of total GSK-3β in skeletal muscle samples from:

A: male wild type (WT) C57B1/6 and transgenic (TG) mice expressing human GSK-3β

B: female wild type (WT) C57B1/6 and transgenic (TG) mice expressing human GSK-3β

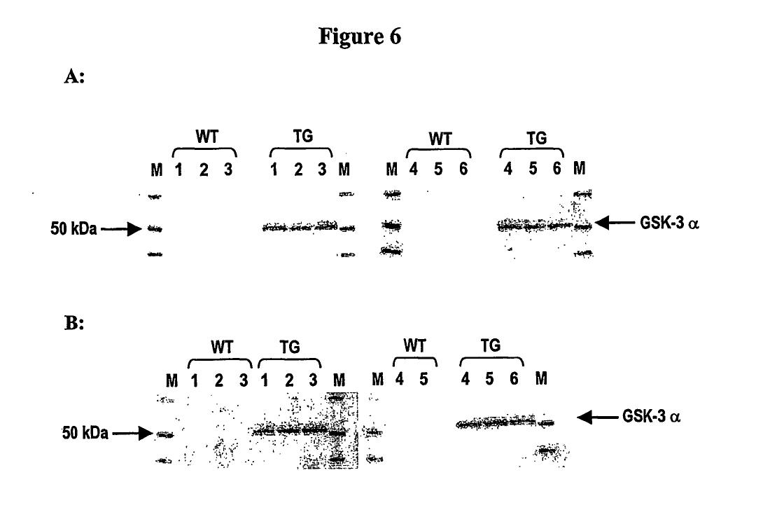

FIG. 6 shows immunoblots of total GSK-3α in skeletal muscle samples from:

A: male wild type (WT) C57BV6 and transgenic (TG) mice expressing human GSK-3α

B: female wild type (WT) C57BV6 and transgenic (TG) mice expressing human GSK-3α

### Example 3

### Phenotypic Effect of Human GSK-3 Overexpression in Transgenic Mice

3.1 Body Weight Measurements and Glucose Tolerance of Transgenics

Male and female mice expressing either human GSK-3α or human GSK-3β in skeletal muscle and age-matched wild-type C57B1/6 mice were housed on a 12 h light cycle with free access to food and water. Measurements of body weight were commenced from 5-6 weeks of age. The GSK-3 Beta transgenic mice and their age matched controls were fed a TEK 2018 (TEK labs) diet. The GSK-3 Alpha transgenic mice and their age matched controls were fed a high protein Purina (5008) diet Oral glucose tolerance tests (OGTT) were performed in overnight fasted mice at the ages indicated in the figure legends. Tail-tip blood was measured at times 0 and then 45, 90 and 135 min following an oral glucose (3 g/kg) load. Blood glucose concentrations were determined and glucose disposal depicted as area under the glucose response curve with time. Areas under the glucose tolerance curves were calculated using the trapezoidal rule.

FIG. 1. shows the effect of overexpression of the human GSK-3 enzymes in mouse skeletal muscle on body weight. Both male and female GSK-3 Beta transgenic mice gained more body weight compared to their age-matched wild type controls, whereas the GSK-3 Alpha transgenic mice weighed less than their respective control mice. Data was taken from 8-13 animals per group for the GSK-3 Beta comparison and 5-6 animals per group for the GSK-3 Alpha comparison, *P<0.05. These results confirm that the GSK-3 enzyme has an effect on body weight, and hence is potentially involved in metabolic diseases, and also highlights a difference between the phenotypic effects of GSK-3α and GSK-3β.

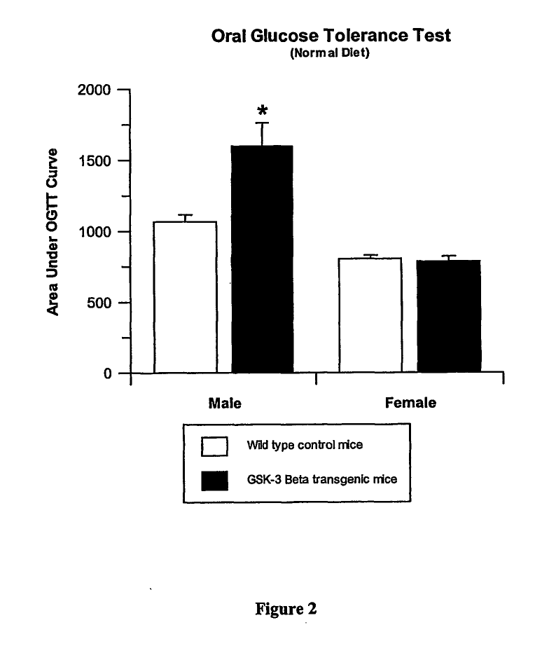

FIG. 2. shows the effect of overexpression of human GSK-3 Beta in mouse skeletal muscle on glucose disposal in 24 week old mice. Glucose disposal, deduced from the area under the OGTR curve, was impaired in male human GSK-3 Beta transgenic mice compared to wild type mice. Data from 8-13 animals per group, *P<0.05.

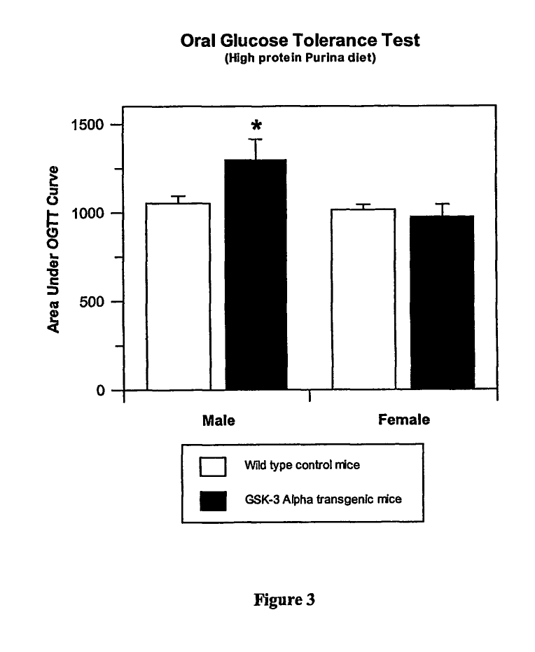

FIG. 3. shows the effect of overexpression of human GSK-3 Alpha in mouse skeletal muscle on glucose disposal in 20 week old mice fed a high protein Purina (5008) diet for 15 weeks. Glucose disposal, deduced from the area under the OGT T curve, was impaired in male human GSK-3 Alpha transgenic mice compared to wild type mice. Data from 5-6 animals per group, *P<0.05.

3.2 Measurement of Glycogen Synthase Activity (GSK3-β Transgenics Only)

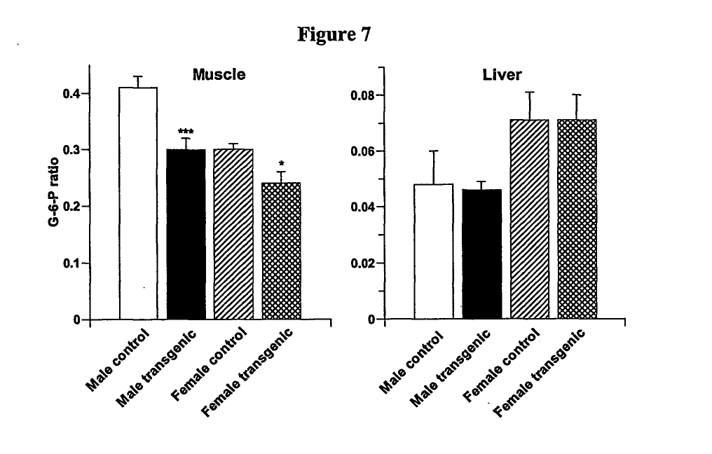

Homogenates of frozen liver and muscle tissue samples were prepared as described for the western blotting (example 2 above), but without the addition of Triton X-100.Samples were centrifuged at 26000 g for 15 minutes at 4° C. and the supernatant isolated, frozen and stored at −80° C. in aliquots prior to assay. The glycogen synthase activity of the muscle and liver supernatants were then measured, in the presence or absence of 20 mM glucose 6-phosphate, as described previously (Cogblan et al (2000) Chemistry & Biology 7:793-803). Results can be seen in FIG. 7 and are expressed as the ratio of glycogen synthase activity in the absence and presence of glucose 6-phosphate. Data are means ±for 8-13 mice per group. *P<0.05, ***P<0.001 for control versus transgenic mice of same age and sex. Glycogen synthase enzyme activity was significantly reduced in the skeletal muscle of both male (−27%) and female (−20%) GSK-3β transgenic mice compared to their respective controls. This is a phenotypic symptom of impaired peripheral glucose metabolism in humans, supporting the fact that these animals will be good models for the screening of compounds to treat metabolic disorders. No significant reduction in hepatic glycogen synthase activity was determined in these mice.

3.3 Blood Lipid Levels

Samples for insulin (0 minutes), NEFA, triglyceride and cholesterol measurement were taken just prior to an oral glucose load in Oral Glucose Tolerance Tests as described in Young et al Diabetes 44 pp 1087-1092 (1995). Plasma insulin levels were also measured 45 minutes after the 3 g/kg oral glucose load. Data are means ±SE for 4-13 mice per group. *P<0.05, **P<0.01, ***P<0.001 for control versus transgenic mice of same age and sex.

| | Insulin | Insulin | | | |
|---|---|---|---|---|---|
| | (0 minutes) | (45 minutes) | NEFA | Triglyceride | Cholesterol |
| | ng/mL | ng/mL | mmol/L | mmol/L | mmol/L |
| Mice, 10 weeks old | | | | | |
| Male Control | 0.43 ± 0.07 | 1.04 ± 0.17 | — | — | — |
| Male GSK-3β transgenic | 1.45 ± 0.27*** | 1.83 ± 0.54* | — | — | — |
| Female Control | 0.24 ± 0.04 | 0.66 ± 0.07 | — | — | — |
| Female GSK-3β transgenic | 0.50 ± 0.09 | 0.85 ± 0.14 | — | — | — |
| Mice, 24 weeks old | | | | | |
| Male Control | 0.45 ± 0.04 | 0.39 ± 0.06 | 2.06 ± 0.27 | 0.63 ± 0.15 | 3.37 ± 0.59 |
| Male GSK-3β transgenic | 0.75 ± 0.14** | 1.10 ± 0.17*** | 3.20 ± 0.28* | 1.70 ± 0.30*** | 6.40 ± 1.14** |
| Female Control | 0.37 ± 0.04 | 0.17 ± 0.04 | 2.04 ± 0.32 | 0.71 ± 0.13 | 1.73 ± 0.19 |
| Female GSK-3β transgenic | 0.41 ± 0.04 | 0.45 ± 0.07* | 2.40 ± 0.22 | 0.89 ± 0.13 | 4.04 ± 0.65** |

Plasma cholesterol levels were significantly increased in both male and female transgenic mice (by 90% and 133% respectively) at 24 weeks of age. Plasma non-esterified fatty acids (NEFA) and triglyceride levels were also significantly increased in the male transgenic mice (by 55% and 170% respectively). These phenotypic characteristics are seen in humans with impaired peripheral glucose metabolism, and also support the fact that these models should be useful for screening compounds to treat metabolic disorders.

3.4 Body Composition

The body composition, total body fat mass and lean tissue mass, of the mice was analyzed at 6 and 25 weeks of age by dual-energy X-ray absoptiometry (DEXA-Lunar Piximus densitometer, Lunar Corp., Wis.). Prior to each session the densitometer was calibrated using a manufacturer supplied QC phantom for bone mineral density and % fat. Mice were anaesthetised with isoflurane (1.5%) during data acquisition; scan cycles were typically completed in 45 min per animal. After completion of the scan, body composition (fat free mass, fat mass and, for the right femur, bone mineral density) for the whole animal was provided by the systems most recent software, version 1.45.

This technique has been previously used successfully to analyse changes in body fat content (adiposity) in mice (25). At 6 weeks of age neither the lean mass or the fat mass of either the male or female transgenic mice were significantly different from their respective controls, even though their total body weights were slightly higher. By 25 weeks of age the fat mass, but not the lean mass, was significantly higher in both the male (by 68%) and female (by 70%) transgenic mice, compared to their respective controls. These results are shown in FIG. 8. Data are means +/− SE of 6-13 mice per group (n=6 for lean mass and fat mass for 6 week old mice). *P<0.05, **P<0.01, ***P<0.0001 for control versus transgenic mice of the same sex.

Terminal tissue weights were also determined in 29 week old transgenic and control mice. The results are shown in FIG. 9. Data are means +/− SE of 6-13 mice per group (n=6 for lean mass and fat mass for 6 week old mice). *P<0.05,**P<0.01, ***P<0.0001 for control versus transgenic mice of the same sex. There was no significant difference in heart weight, but the weight of the liver, and the subcutaneaous white and interscapular brown adipose depots were increased in both the male and female transgenic animals. Perigenital adipose tissue weights were also significantly increased in the female transgenic mice. These results suggest that an increase in fat mass, rather than lean body mass, therefore accounts for the increased weight of the GSK-3β transgenic mice compared to their respective controls.

### EXAMPLE 4

### Reversal of Phenotype by a GSK-3 Inhibitor.

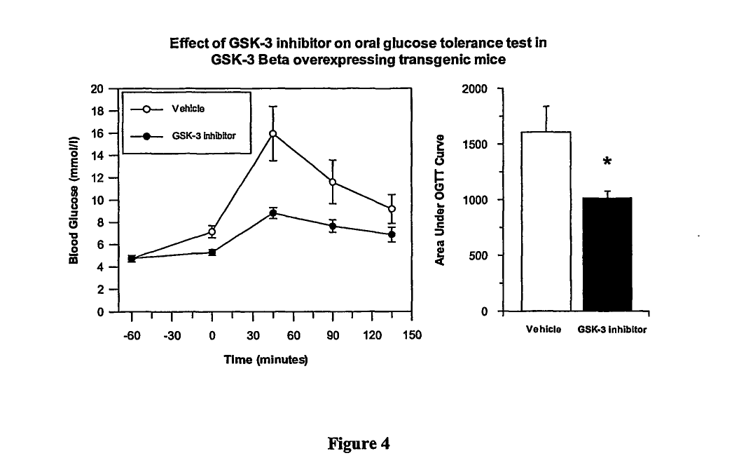

In order to demonstrate that the phenotypes generated by overexpression of the GSK enzyme could be reversed, a GSK-inhibitor (300 umol/kg) was administered by oral gavage to 28 week old male GSK-3β overexpressing transgenic mice. FIG. 4 shows the results. Glucose disposal, deduced from the area under the OGTT curve, was improved in male human GSK-3 Beta transgenic mice treated with the GSK-3 inhibitor. Data from 4 animals per group, *P<0.05.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

1. A transgenic non-human mammal whose genome comprises a polynucleotide encoding at least one human Glycogen Synthase Kinase-3 polypeptide under the control of a regulatory sequence which specifically directs expression of said at least one GSK-3 polypeptide in muscle tissue.

2. A transgenic non-human mammal according to claim 1 wherein at least one of said human Glycogen Synthase Kinase-3 polypeptides is the Alpha isoform of the enzyme.

3. A transgenic non-human mammal according to claim 2 wherein the polynucleotide encoding the GSK-3α polypeptide is selected from the group consisting of:
(a) a polynucleotide comprising a polynucleotide sequence having at least 95%, 96%, 97%, 98%, or 99% identity to of SEQ ID NO:1 or SEQ ID NO:5;
(b) a polynucleotide comprising SEQ ID NO:1 or SEQ ID NO:5;
(c) a polynucleotide having at least 95%, 96%, 97%, 98%, or 99% sequence identity to SEQ ID NO:1 or SEQ ID NO:5;
(d) a polynucleotide of SEQ ID NO:1 or SEQ ID NO:5;
(e) a polynucleotide encoding a polypeptide sequence having at least 95%, 96%, 97%, 98%, or 99% sequence identity to SEQ ID NO:2;
(f) a polynucleotide which encodes a polypeptide of SEQ ID NO:2;
(g) a polynucleotide which encodes a polypeptide sequence having at least 95%, 96%, 97%, 98%, or 99% sequence identity to SEQ ID NO:2;
(h) a polynucleotide having or comprising a polynucleotide sequence that has an Identity Index of 0.95, 0.96, 0.97, 0.98, or 0.99 compared to SEQ ID NO:1;
(i) a polynucleotide having or comprising a polynucleotide sequence encoding a polypeptide sequence that has an Identity Index of 0.95, 0.96, 0.97, 0.98, or 0.99 compared to SEQ ID NO:2.
(i) a polynucleotide fragment of SEQ ID NO:1 or SEQ ID NO:5 that encodes a functional GSK-3 polypeptide; or
(k) a polynucleotide that will hybridise under stringent conditions to a polynucleotide of SEQ ID NO:1 or SEQ ID NO:5 and that encodes a functional GSK-3 polypeptide.

4. A transgenic non-human mammal according to claim 3 wherein said polynucleotide encodes the polypeptide of SEQ ID NO:2

5. A transgenic non-human mammal according to claim 1 wherein at least one of said human glycogen synthase kinase-3 is the beta isoform of the enzyme.

6. A transgenic non-human mammal according to claim 5 wherein the polynucleotide encoding the GSK-3β polypeptide is selected from the group consisting of:
(a) a polynucleotide a comprising a sequence having at least 95%, 96%, 97%, 98%, or 99% identity to SEQ ID NO:3 or SEQ ID NO:6;
(b) a polynucleotide comprising SEQ ID NO:3 or SEQ ID NO:6;
(c) a polynucleotide having at least 95%, 96%, 97%, 98%, or 99% sequence identity to SEQ ID NO:3 or SEQ ID NO:6;
(d) a polynucleotide having SEQ ID NO:3 or SEQ ID NO:6;
(e) a polynucleotide comprising a polynucleotide sequence encoding a polypeptide sequence having at least 95%, 96%, 97%, 98%, or 99% identity to SEQ ID NO:4;
(f) a polynucleotide comprising a sequence encoding a polypeptide of SEQ ID NO:4;
(g) a polynucleotide having a sequence encoding a polypeptide sequence having at least 95%, 96%, 97%, 98%, or 99% identity to SEQ ID NO:4;
(h) a polynucleotide having a sequence encoding a polypeptide of SEQ ID NO:4;
(i) a polynucleotide having or comprising a polynucleotide sequence that has an Identity Index of 0.95, 0.96, 0.97, 0.98, or 0.99 compared to SEQ ID NO:3 or SEQ ID NO:6;
(j) a polynucleotide having or comprising a polynucleotide sequence encoding a polypeptide sequence that has an Identity Index of 0.95, 0.96, 0.97, 0.98, or 0.99 compared to SEQ ID NO:4;
(k) a polynucleotide fragment of SEQ ID NO:3 or SEQ ID NO:6 that encodes a functional GSK-3 polypeptide; or
(l) a polynucleotide that will hybridise under stringent conditions to SEQ ID NO:3 or SEQ ID NO:6 and that encodes a functional GSK-3 polypeptide.

7. A transgenic, non-human mammal according to claim 6 wherein said polynucleotide encodes a polypeptide of SEQ ID NO:4

8. A transgenic, non-human mammal according to claim 1 wherein the genome of said mammal comprises (a) a polynucleotide sequence encoding an alpha isoform of the human glycogen synthase kinase polypeptide and (b) a polynucleotide sequence encoding a beta isoform of the human glycogen synthase kinase polypeptide.

9. A transgenic non-human mammal according to claim 1 wherein said animal is a rodent.

10. A transgenic rodent according to claim 9 wherein the rodent is a mouse.

11. The transgenic non-human mammal according to claim 1 wherein the said regulatory sequence comprises a skeletal muscle-specific promoter.

12. A transgenic non-human mammal according to claim 11 wherein the skeletal muscle-specific promoter is alpha actin.

13. A transgenic non-human mammal according to claim 1 which exhibits impaired glucose tolerance.

14. A transgenic non-human mammal according to claim 1 which exhibits increased or decreased weight.

15. (Canceled)

16. A method of producing a transgenic non-human mammal comprising introducing into said non human mammal a transgene construct comprising a polynucleotide encoding a human GSK3 polypeptide and a regulatory sequence which specifically directs expression of said GSK3 polypeptide.

17. A method of producing a transgenic non-human mammal according to claim 16, wherein the transgene construct is introduced into mouse Embryonic Stem cells, using electroporation, retroviral vectors or lipofection.

18. A method of producing a transgenic non-human mammal according to claim 16 comprising pronuclear injection of the transgene construct into egg cells.

19. A method of screening a test agent agents for use in the treatment of a disorder characterised by a change in glucose homeostasis, which method comprises administering said test agent to a transgenic non-human mammal of claim 1, and measuring the effect of said test agent on glucose homeostasis.

20. A kit for screening agents for use in the treatment of a disorder characterised by a change in glucose homeostasis, which kit comprises a transgenic non-human mammal as defined in claim 1.

21.-26. (Cancelled)

27. A transgene comprising a polynucleotide encoding human GSK-3 polypeptide operably linked to a muscle specific regulatory sequence.
